(19) United States
(12) Patent Application Publication
(10) Pub. No.: US 2007/0090009 A1
(43) Pub. Date: Apr. 26, 2007

(54) Packaging and packaging process for compressible articles

(75) Inventors: Timothy Patrick Clare, Appleton, WI (US); Duane Lyle McDonald, Neenah, WI (US); Andrew Mark Long, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/254,623

(22) Filed: Oct. 20, 2005

(51) Int. Cl. A61B 19/02

(52) U.S. Cl. 206/440

(57) ABSTRACT

The present invention provides a compressed package of articles having a packaging component with a seal, and a plurality of compressible articles in an array having an uncompressed length. The compressible articles are compressed to a first compressed state having a first compressed length and disposed within the packaging component in the first compressed state. The packaging component retains the plurality of compressible articles in the first compressed state, and the packaging component and the plurality of compressible articles in the first compressed state form a package having a first package volume. Vacuum compression is used to compress the packaging component and the plurality of compressible articles in the first compressed state disposed in the packaging component together to a second compressed state having a second package volume. The array of compressible articles has a second compressed length after the vacuum compression. The package is sealed with a seal such that the seal holds the package containing the plurality of compressible articles in the second compressed state. As a result of the two compressions, the second compressed length of the array is less than the first compressed length of the array. Likewise, the first compressed length is less than the uncompressed length. Also, disclosed is a process for producing the package of compressed articles.

### FIELD OF THE INVENTION

The present invention relates generally to packaging and a packaging process for compressible articles. More particularly, the present invention relates to packaging and a packaging process for absorbent personal care articles.

### BACKGROUND OF THE INVENTION

Typically, retailers allocate a set amount of shelf space to manufacturers so that the manufacturer of products may display and market their products within the retail store. These allocations of shelf space are substantially fixed over short periods of time, but change due to various factors such as the net sales of a given product or the net sales of products from a given manufacturer. This is especially true for large discount retailers. As a result, a manufacturer is typically not given enough shelf space to display and market all of their products or provide a variety of product counts for a given product. Therefore, there is a need in the art to provide a way for a manufacturer of products to reduce the size of the packaging of the products to more efficiently use allocated shelf space in a retail store to provide a variety of products and packages of products in a variety of product counts or package sizes.

In addition, distribution and transportation costs continue to rise. As a result, it becomes more expensive for manufacturers of bulky products to transport the bulky products from the place of manufacture or a manufacturer's warehouse to the distribution center of a retailer, or to a retail store. This often leads to increased cost being added to the cost of the product, which is generally passed onto the consumer by increasing the price of these bulky products. Often, bulky items, such as diapers and incontinence articles, such as disposable underwear, are compressed to a certain degree when packaged to reduce the overall volume of the packages. With reduced volume, more packages can be transported from the place of manufacture or the manufacture's warehouse in a given transportation vehicle, such as a trailer truck, or train car. There is a need in the art to reduce distribution and transportation costs of transporting bulky products to retailer.

Bulky consumer products also present a storage problem for consumers. In many older homes, and relatively cramped living quarters, such as college dorms, small apartments and the like, closed storage is often only available on a limited basis. As a result, consumers who live in these types of living arrangements prefer that bulky consumer products be as small as possible, but at the same time prefer that the bulky consumer products are easy to remove from the packaging.

Current methods of reducing the volume of packages of bulky articles have a number of drawbacks. For example, vacuum packing of bulky products allows the volume of the package to be greatly reduced; however, when the seal of the vacuum seal of the package is broken, the bulky product will expand to their original size. This will often result in problems for consumers with limited storage space. In addition, the amount of packaging material needed for vacuum packing often increases the cost of the packaging, thereby offsetting any cost advantage obtained in transportation and distribution of the consumer products. Other methods of reducing volume of bulky consumer products are to mechanically compress the consumer products prior to placing the consumer product into a package. When the package is opened, unlike the vacuum packed package, the products will not expand to their original uncompressed size since the packaging will keep the products in a compressed state. Mechanical compression has limits. It has been discovered that consumer products containing fibrous materials, such as diapers, incontinence articles and the like, will become damaged and will not perform as intended if the mechanical compression reduces the size of the consumer products by more than about 30% by volume. There is a need in the art to provide an effective way to reduce the volume of packages of bulky consumer products without the drawbacks of the current methods of reducing volume.

### SUMMARY OF THE INVENTION

Generally stated, the present invention provides a compressed package of articles. The compressed package has a packaging component having a seal, and a plurality of compressible articles in an array. The compressible articles have an uncompressed length. The compressible articles are compressed to a first compressed state. This first compressed state has a first compressed length and disposed within the packaging component in the first compressed state. The packaging component retains the plurality of compressible articles in the first compressed state, and the packaging component and the plurality of compressible articles in the first compressed state form a package having a first package volume. Vacuum compression is used to compress the packaging component and the plurality of compressible articles in the first compressed state disposed in the packaging component to a second compressed state having a second package volume. The array of compressible articles has a second compressed length after the vacuum compression. The package is sealed with the seal such that the seal holds the package containing the plurality of compressible articles in the second compressed state. As a result of the two compressions, the second compressed length of the array is less than the first compressed length of the array. Likewise, the first compressed length is less than the uncompressed length.

In another embodiment of the present invention, the compressible articles in the first compressed state are compressed to the first compressed state by mechanical compression. With the package component holding the array of compressible articles in the first compressed state, the seal provided to hold the package and array of compressible articles in the second compressed state may be removed and the package containing the plurality of articles in the second compressed state and the package will return to the first package length.

In another embodiment of the present invention, provided is a package having an outer package containing one or more of the compressed packages of the present invention disposed therein.

In yet another embodiment of the present invention, provided is a method of packaging a plurality of compressible articles. The method of this embodiment of the present invention includes providing a packaging material; and providing an array of compressible articles. The array of compressible articles contains a plurality of compressible articles and the array has an uncompressed length. The method also includes mechanically compressing the array of compressible articles to form a compressed array of compressible articles having a first compressed length; forming a packaging component from the packaging material; and placing the compressed array of compressible articles into the packaging component. The package component and the compressed array of compressible articles in the packaging component are together compressed by subjecting the compressed array of compressible articles in the packaging component to a pressure lower than atmospheric pressure. The array of compressible articles, after the vacuum compression, has a second compressed length. Next, sealing the packaging component occurs by placing a seal on the package thereby forming a sealed package of a compressed array of compressible articles.

### BRIEF DESCRIPTION OF THE DRAWINGS

FIG. 1 shows an array of compressible articles in an uncompressed state.

FIG. 2 shows an array of compressible articles compressed to a first compressed state.

FIG. 3 shows an array of compressible articles compressed to a second compressed state.

FIG. 4 shows an exemplary process which may be used to form the compressed package of articles of the present invention.

FIG. 5 shows an array of compressible articles placed in a form fill and seal package wherein the array of compressible articles is in a first compressed state.

FIG. 6 shows the array of compressible articles after vacuum compression to a second compressed state.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As used herein, the term “array” or “array of compressible articles” is intended to mean a series of at least two articles or compressible articles placed in the same orientation with each other. Each article in the array has a front surface and a back surface and is positioned in the array relative to each other such that the first article has its back surface adjacent the front surface of the next article in the array. The array of compressible articles may be positioned either horizontally or vertically. It is not critical to the present invention in which direction the articles are arranged in the array; however, the compression of the articles must be at least in the direction such that the length of the articles in the array is reduced when compressive force is applied. When there are more than two articles in the series, at least one article is adjacent to another article on at least two sides. The number of articles in the series which are adjacent at least two other article is equal to the total number of articles in the series minus 2. That is, when the series of articles that form the array is greater than two, each article in the series is between two other articles in the series, except for the first article in the series and the last article in the series.

As used herein, the term “array length” refers to the distance between the first article in the array to the last article in the array. It should be pointed out that the term “length” as used herein is not intended to be limited to a distance in the vertical direction. Length is meant to be the distance in the direction of the array of the articles.

As used herein, the term “second compressed length” is intended to mean the distance between the outside surface of the first article in a row or array of compressible articles aligned in the same direction to the outside surface of the last article in the row or array of compressible articles when a second compressive force is applied to the array of compressible articles in a first compressed state in a direction from the outside surface of the first article in a direction towards the outside surface of the last article in the row or array of compressible articles.

It should be understood that the term “personal care product” or “personal care article” as used herein refers to any article used to control bodily fluids, and includes “absorbent products,” which refers to any article configured to absorb and retain bodily exudates, including urine, bowel movements, blood and menses, and includes such a product in a packaged and unpackaged configuration. As such, “personal care products” as used herein, includes without limitation, diapers, child toilet training pants, adult incontinence garments, male incontinence products, tampons, vaginal suppositories, pantiliners, pads, sanitary napkins, tissues, wipes, etc. For example, personal care products include, without limitation, Poise® feminine care products, including pantiliners and pads, and Kotex® feminine care products, including sanitary napkins, and liners, Huggies® baby and child care products including diapers, disposable swim diapers, children's training pants and the like, all available from Kimberly-Clark Corporation, Neenah, Wis.

### DETAILED DESCRIPTION OF THE INVENTION

In order to obtain a better understanding of the present invention, attention is directed to the Figures. In the present invention as is shown in FIG. 1, the array of compressible articles 10 have an uncompress length UCL. As used herein, the term “uncompress length” is intended to mean the distance between the outside surface 22 of the first article 21 in a row or array of compressible articles 10 aligned in the same direction to the outside surface 24 of last article 23 in the row or array of compressible articles 10 without any compressive forces being applied to the row or array of compressible articles. As used herein, without any compressed forces, means that no additional force, other than atmospheric pressure is applied to the array of compressible articles in any direction. That is, the term uncompressed length is intended to mean a length of the array of compressible articles 10 as the array occurs under atmospheric pressure without an external force being applied to the array 10. As an alternative, if the array of compressible articles 10 is stacked in a vertical direction (not shown) i.e. the first article is below the last article or visa versa, the weight of the articles may result in some compression of the articles. If in a vertical stack, the weight of the articles which may result in some compression of articles placed lower in the stack. This compression is not considered an external compressive force and the height or length of the array articles in the stack is the UCL, even though the height of the array of compressible articles may be smaller than the horizontal length.

In the present invention, the uncompressed array of compressible articles 10 is compressed to first compressed length FCL. As used herein, the term “first compressed length” is intended to mean the distance between the outside surface 22 of the first article 21 in a row or array of compressible articles 10 aligned in the same direction to the outside surface 24 of last article 23 in the row or array of compressible articles 10 when a first compressive force 13 is applied to the array of compressible articles 10 in an uncompressed state in a direction from the outside surface 22 of the first article 21 in a direction towards the outside surface 24 of the last article 23 in the row or array of compressible articles. The first compressed length FCL is a percentage of uncompressed length. Once the array of compressible articles are compressed to the first compressed length FCL, which is also referred to herein as the first compressed state, the array of article 10 is placed in a packaging component 30, shown in FIG. 4, which will be described in more detail below. The packaging component holds and helps maintain the array of compressible articles 10 in the first compressed state such that the array of compressible articles in the package still has the first compressed length.

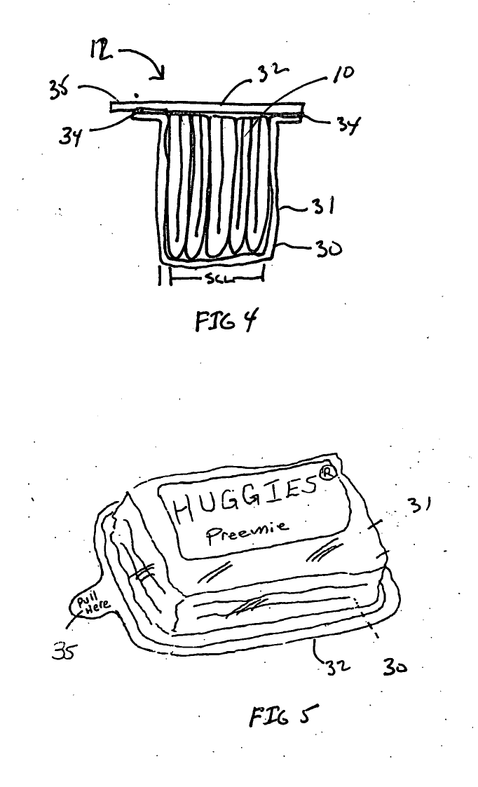

Once in the array of compressible articles 10 are in the first compressed state the array of compressible articles 10 are again compressed using a vacuum compression process. This vacuum compression process further compresses the array of compressible articles 10 in the packaging component 30 to a second compressed length SCL. As used herein, the term “second compressed length” is intended to mean the distance between the outside surface 22 of the first article 21 in a row or array of compressible articles 10 aligned in the same direction to the outside surface 24 of last article 23 in the row or array of compressible articles when a second compressive force 15 is applied to the array of compressible articles 10 in a first compressed state. This is shown in FIG. 3. The second compressed length SCL is a percentage of the first compressed length FCL and a percentage of the uncompressed length UCL of the array of compressible articles. The second compressed length SCL is a smaller percentage of the uncompressed length UCL than the first compressed length FCL. Since vacuum compression is used as the second compressive force 15, an additional compressive force 17 is also applied to the array of compressible articles in a direction which is perpendicular to the compressive force 15.

In the present invention an array of compressible articles are contained within a packaging component. To obtain a better understanding of the invention, attention is directed to FIG. 4, which shows a possible embodiment of the present invention. In FIG. 4, the array of compressible articles 10 are contained within a package 30. As shown in FIG. 4, the package 30 has two portions, a first portion 31 and a second portion 32. Suitable materials in which the package may be prepared include materials which can be sealed and have a low gas permeability. In addition, the package should be able to hold the array of compressible articles in the first compressed state without breaking and without excessive bulging or stretching of the material. Examples of such materials include, but are not limited to, a polymeric film, such as, by way of non-limiting example, a polyethylene terephthalate (PET), a polyvinyldichloride (PVDC), having an oxygen transmission of about less than 3.0 cc/100 in²/24 hours @ 73° F. and 0% relative humidity (RH). Multilayer films, each providing a different function, may be utilized. For example, one layer may have a heat sealable property, such as polypropylene or polyethylene, another layer may provide strength, such as polyester and/or nylon, and another layer may provide substantially reduced gas permeability, such as PET or PVDC. Also, films with reduced permeability to vapors (that is, films that have substantially low gas permeability or are substantially gas impermeable for a variety of applications) may be composites, such as where one layer is a flexible polymer, such as polyurethane, polyethylene, ether polyurethane, or polypropylene, while another layer is coated on or coextruded and serves as a barrier layer. Barrier layers can generally be viewed as substantially organic based or substantially inorganic based. For example, U.S. Pat. No. 3,442,686, hereby incorporated by reference herein, describes a film composite in which silicon oxide coatings are deposited on polymers to serve as a barrier layer. This produces barrier coatings on even quite thin polymer films of oxygen transmission rate properties of about 0.2 cc/100 in²/day and similar water vapor transmission rate properties. However, it will be appreciated that any material having substantially low gas permeability or substantially gas impermeable material(s) known in the art may be used. Other materials may also be used including laminates of films with materials such as nonwoven webs. By using laminates with the nonwoven material on the outside, a more attractive package may be formed, having a softer fell and appearance.

Desirably, with a substantially low gas permeable material and/or package, the gas permeability rate is less than about 5.0 cc/100 in²/24 hours to about 0.05 cc/100 in²/24 hours. More desirably, the gas permeability rate is less than 4.0 cc/100 in²/24 hours to about 0.07 cc/100 in²/24 hours. Even more desirably, the gas permeability rate is less than about 3.0 cc/100 in²/24 hours to about 0.10 cc/100 in²/24 hours. Even more desirably, the gas permeability rate is less than about 2.0 cc/100 in²/24 hours to about 0.10 cc/100 in²/24 hours.

In addition to the gas permeability, the packaging material has a seal which holds the vacuum applied to the packaging 30 and the array of compressible articles 10. The seal is critical to the present invention since seal prevents air or moisture from entering the package, thereby keeping the package in the second compressed state. The seal is created by sealing the package 30 using a known sealing method. Any sealing method can be used, including adhesive sealing, ultrasonic sealing, heat sealing, stitching and the like, provided that the seal created will allow the package to retain a vacuum within the package. In addition, a combination of sealing methods can also be used. The only requirement of the sealing method is that the seal resulting from the sealing method will hold the package 30 and the array of compressible articles 10 in the second compressed state. The seal should be strong enough that when the package is removed from the vacuum chamber, discussed below, that the seal is not broken or otherwise compromised, such that the vacuum created within the packaging is lost. In addition, the seal should be strong enough that the seal is not broken or otherwise compromised during transportation to the and from the retail store or warehouse, while being displayed in a retail store or at a consumer place of use, until the consumer is ready to open the package. Typically, the seal will have a seal strength of at least 0.75 lb_{f} to about 1 lb_{f}, measured as a tensile strength. The seal can readily be broken by the consumer when the use of the articles contained in the package is needed or desired. Furthermore, a package of the present invention can have one or more seals, provided that each of the seals is durable and at least one of the seals can be readily broken by a consumer or user of the articles.

The entire package can be prepared from a single piece of packaging material or can be prepared from two or more pieces of material. It is not critical to the present invention how many separate pieces of material are used to prepare the package. In one embodiment of the present invention, two pieces of material are used to prepare the package. In this configuration, a seal is created between two pieces of material. In this regard, attention is directed to FIG. 4, which shows an embodiment of sealed package 12 of the present invention containing an array of compressible articles 10 are within a packaging component 30. The packaging component has a first portion 31 and a second portion 32. A seal 34 is created between the first portion 31 and the second portion 32 of the packaging component. The first portion 31 of the package maintains the array of article in the first compressed state when the seal is broken and the vacuum created with the package is released. To maintain the vacuum of within the package 30 containing the array of compressible articles 10, the seal is desirable air impermeable. As an alternative, the first portion 31 of the package 30 and the second portion of the package 32 may be prepared from a single piece of material, provided that a package can be formed which will retain a vacuum.

The compressed package of the present invention has a packaging component having a seal, and a plurality of compressible articles in an array. The compressible articles have an uncompressed length UCL in an uncompressed state, a first compressed length FCL in a first compressed state. The packaging component retains the plurality of compressible articles in the first compressed state, and the packaging component and the plurality of compressible articles in the first compressed state form a package having a first package volume. At this point, the package is not completely sealed so that package can be placed in a vacuum. Once in the vacuum, the package is sealed with a seal that can be broken by a user. At this point, the array of compressible articles is sealed within the package component and the seal on the package component provides a package component which is essentially air impermeable. Once sealed, the vacuum is released on the package with the array of compressible articles and the package and array of article is exposed to atmospheric pressure. The atmospheric pressure places an external force on the package, causing the package and array of compressible articles within the package to be compressed. This second compression is referred to herein as vacuum compression. Vacuum compression is used to compress the packaging component and the plurality of compressible articles in the first compressed state disposed in the packaging component together to a second compressed state having a second package volume. The array of compressible articles has a second compressed length after the vacuum compression. The vacuum sealed package will retain the articles in the second compressed state until the seal or the package material itself is compromised such that the vacuum condition within the package is released. As a result of the two compressions, the second compressed length of the array is less than the first compressed length of the array and the first compressed length is less than the uncompressed length. Also, the second package volume is less than the first package volume.

In the present invention, so long as the vacuum condition is retained, the package and the array of compressible articles will remain in the second compressed state. When the vacuum is released, the package and the array of compressible articles will return to the first compressed state, where the packaging material will hold the compressible articles in the first compressed state. By having the package of the present invention twice compressed, where the first compression will be held by the packaging component, the size of the package of the array of compressible articles can be drastically reduced for shipping, for display in a retail store, and for storage by the consumer. In addition, by having the first compression held by the packaging component, the size of the package will still be reduced compared to a package which is only vacuum packed. That is, the first compressed state has a smaller volume than the uncompressed array of articles or a package containing an uncompressed array of articles. As a result, the package in the first compressed state takes less storage room than a package which is solely vacuum packaged. In addition, once the vacuum is released, the package of the array of compressible articles has a size which is about the same as current compressed packages containing an array of compressible articles.

In the present invention, the compressed package of articles is compressed to a first compressed length using a known compression means. Generally, the first compressed length of the array is approximately between about 60% and about 95% of the uncompressed length of the array. Desirably, the first compressed length of the array is approximately between about 65% and about 80% of the uncompressed length of the array. In addition, generally the second compressed length is approximately between about 50% and 90% of the first compressed length of the array. From a cost and efficiency standpoint, the second package length of the array is between about 60% and about 80% of the first compressed length of the array. As a result, the second compressed length of the array is between about 40% and about 60% of the uncompressed length of the array.

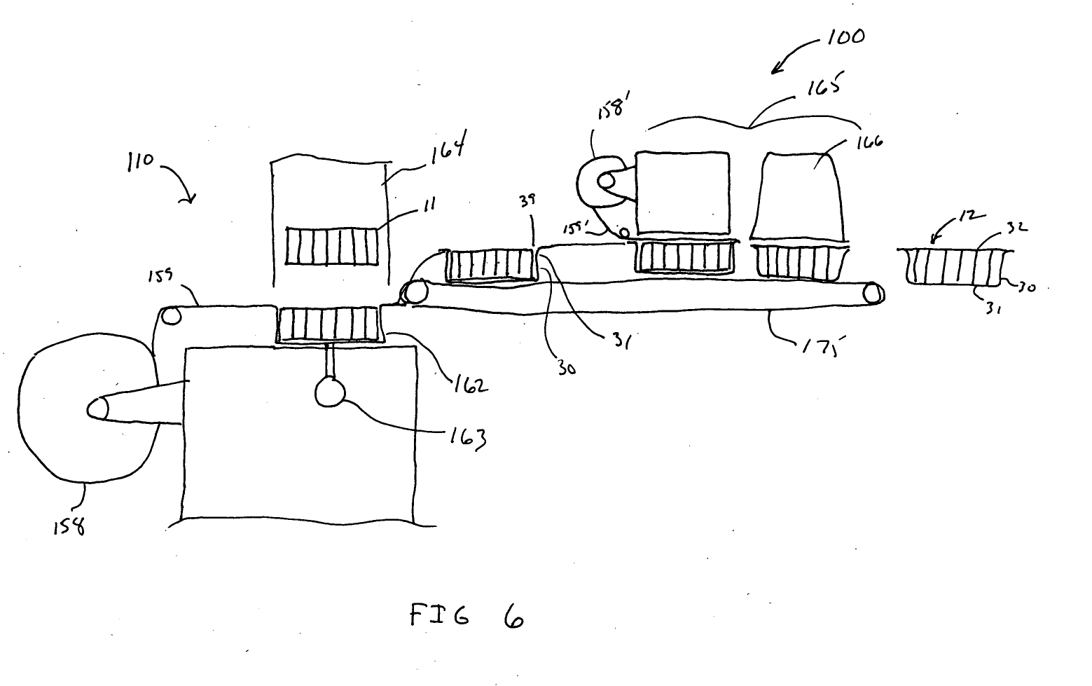

In the present invention, when seal 34 of the package 30 is broken by the user of the articles contained within the package, the package and articles in the package expand from the second compressed state and return to the first compressed state. The seal 34 can be broken in a variety of ways, including cutting the package material or by providing an opening element for the user to break the seal. Desirably, the opening element should provide the user with a mechanism to open the package at the seal with a controlled opening. The controlled opening may allow the user to create an opening in the package for releasing the vacuum and to access one or more of the articles in the array of compressible articles. Desirably, the opening element should allow the user to open the package without having to separate the package into two unconnected portions. Examples of possible opening elements include, for example, providing perforation outside of the seal area which led toward the seal area to aid the user to tear the package outside the seal area towards the seal area to release the vacuum when the seal is compromised. Another example of a opening device which may be used includes a pull tab. Further desirably, as illustrated in FIG. 5, the package 30 will have a pull tab 35. The pull tab 35 desirably extends beyond a perimeter 36 of the package 30, so that it is easy to grasp between a thumb and a finger by a caregiver, and permits easy opening of the package while maintaining the package 30 in one piece after being opened, and after the article has been removed.

The pull tab 35 shown in FIG. 5 may be formed as a portion of the top portion 32 of the package 30, and is desirably, but not by way of limitation, not coterminous with the bottom portion 31 of the package 30 to which the top portion is sealed. The pull tab 35 may have pull indicia such as words, symbols, and so forth (for example, but not by way of limitation, “Pull Here” illustrated in FIG. 5). The pull tab 35 may be formed of a thicker material, or may be embossed or otherwise textured, and so forth, to provide easy grasping and pulling by a caregiver. The pull tab 35 may be formed, in one or more corners of the package 30, or one or more pull tabs 35 may be provided on any portion(s) of the package 30 (not shown). Desirably, the edges of the pull tab 35 are curvilinear.

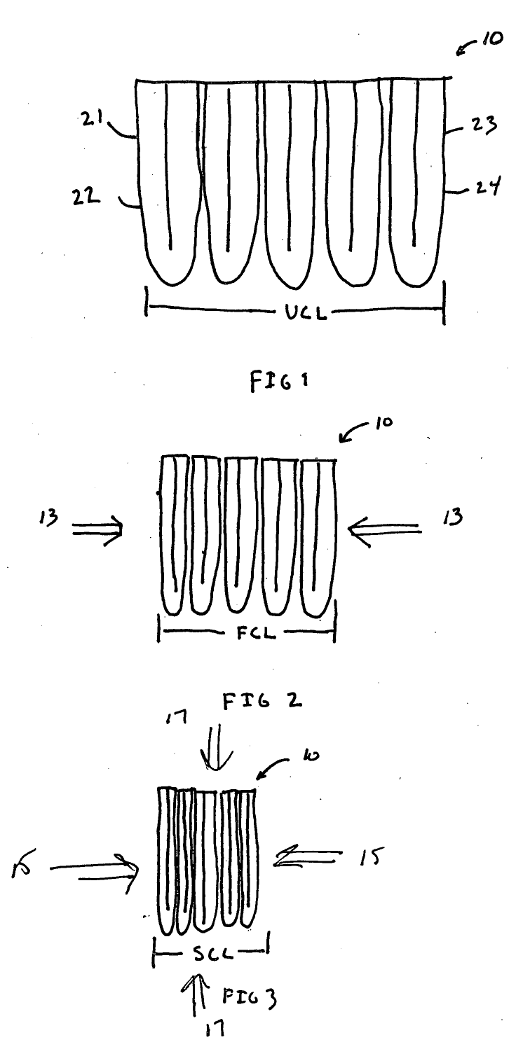

To aid understanding of the present invention, the process in which the compressed package of absorbent article may be prepared will be described in detail. In this regard, attention is directed to FIG. 6, which shows an exemplary process 100 which may be used to prepare the compressed package of compressible articles 12 of the present invention. The articles to be stored and compressed in the package 30 of the present invention are prepared into an array of compressible articles 10. The articles are compressed to a first compressed state to form an array of compressed articles in a compressed state 11. The array of compressible articles are compressed in the direction of the series of articles, as is shown in FIG. 2, by applying a force to both sides of the array 10 to a first compressed state. Any known method of compressing the array of articles to the first compressed state may be used. As an example, mechanical compression may be used.

Once in the first compressed state, the array of compressed articles 11 is transferred to the packaging apparatus 110 for packaging and further compression. An exemplary packaging apparatus 110 has a first reel 158 of sheet material 159, which is used to form packaging material, is advanced from the reel 158 to a vacuum cavity former 162. In the vacuum cavity former 162, a vacuum 163 is applied to the sheet material. In addition, heat may be optionally applied to the sheet material. The sheet material takes the shape of the cavity of the vacuum former 162. The optional heating of the sheet material in the vacuum cavity former aids to thermoform the sheet material to the shape of the cavity, thereby forming the sheet material into a three dimensional package, which is sized to receive that array of compressed articles 11 from the loading device 164. The array of articles in the first compressed state 11 is transferred to the sheet material 159 at the vacuum cavity former from a loading device 164. The loading device 164 may be provided with a means to compress array of compressible articles 10. In the alternative, the loading device 164, keeps the already compressed array of articles 11 in the compressed state until the array of compressed articles 11 are placed in the formed package. The vacuum cavity former 162 essentially forms that first portion 31 of the package. The size and shape of the shaped package 30 can be varied depending on the size, type, number shape and degree of compression of the compressible article in the first compressed state. In the present invention, it is desirable that the shaped package 30 is approximately a length, width and a depth which is roughly the same as the length, width and depth of the array of compressible articles in the first compressed state, so that the array of compressible articles 10 are retained in or about the first compressed state. Once formed, the package 30 in the vacuum cavity former is filled with the array of compressed articles 11 in the first compressed state. The loading device 164 releases the array of compressed articles 11 and the shaped package 30 retains the array of compressible articles in the first compressed state or slightly less compressed than the first compressed state.

The array of compressed articles 11 with the packaging component contained therein is carried to a packaging station 165. The compressed articles 11 in the lower portion of the package 31 are carried into the packaging station 165 by a conveyor 175 or other similar transporting device. The packaging station 165 has a vacuum sealing chamber 166, which evacuates air from the package of articles 30 and seals the package. The packaging station 165 also contains second roll 158′ of a sheet material 159′ is fed to the packaging station and is used to form the second portion of the package 32. The sheet material 159 is passed over the open or top portion 39 of the package 30 which seals the compressed articles 11 in the package 30 while package 30 and compressed article 11 are in the vacuum chamber 166. A vacuum is applied to the array of compressed articles 11 and the lower portion of the package 31, and sheet material 158′ is positioned and sealed over each lower portion of the package 31 via sealing devices, such as thermal sealing, ultrasonic bonding, or any other sealing methods known by those skilled in the art. The vacuum depressurizes the package 30 and the array of compressed articles 11 in the package to a pressure which is less than the atmospheric pressure outside of the package 30. After the package is sealed, the vacuum is removed from the packaging station 165 and moved down the assembly line (not shown) for further packaging.

It will be appreciated that many techniques and apparatus for vacuum packaging are known to those skilled in the art, and are commercially available. Any type of packaging device may be utilized which permits the article 10 to perform in the manner described and illustrated herein. Another type of vacuum packaging is disclosed, for example, but not by way of limitation, in U.S. Pat. No. 4,833,862 to Bortolani, et al., which is hereby incorporated by reference in its entirety. Many techniques and apparatus for vacuum packaging are known in the art and commercially available.

The amount of vacuum is an amount necessary to result in compression of the articles from the first compressed state to the second compressed state, thereby reducing in size the articles and the packaging materials. Care should be taken when using the vacuum, since excessive vacuum may destroy the elements of the articles, thereby disrupting the articles' functionality. For example, when the article are absorbent articles, containing a cellulosic material pulp or superabsorbent particles, the compression should be such that the compression displaces the superabsorbent particles or overly compresses the cellulosic material. The amount of vacuum is generally in a range of about 32 to about 5 inches of Mercury, typically in the range of about 30 to about 8 inches of Mercury. Desirably, the range is about 28 to about 10 inches of Mercury and most desirably in the range of about 25 to about 20 inches of Mercury.

A number of different packages may be utilized which are suitable for use with the present invention. Types of packages which are particularly suitable include, but are not limited to, form-fill-seal packages, blister packages, injection molded packages (with and without living hinges), heat sealed pouches, thermally formed trays with lids, and any suitable package known to those skilled in the art.

The articles contained within the package can be any article which may be compressed and arranged in array. An example of articles that may be packaged in the package of the present invention includes personal care articles. Personal care articles include, for example, diapers, training pants, adult incontinence pads, adult incontinence garments, sanitary napkins, pantiliners and other similar products.

When packages are vacuum compressed, the package tends to become highly wrinkled and may have another appearance which may be undesirable from an aesthetic standpoint, which may cause consumers not to purchase the package. To solve this perceived problem, the vacuum sealed package of the present invention may be wrapped in an additional wrapper. The additional wrapper material may be prepared from the same package material. In addition, other materials may be used including paper, paperboard, cardboard, nonwoven materials, films, laminates of these materials or other similar materials. When an outer packaging material, two or more of the twice compressed packages may be wrapped or contained within the outer packaging material. By providing two or more of the twice compressed packages in the outer wrapper, one of the packages may have the seal released, while the other package or packages are retained in the second compressed state until the articles in the second package are needed. This will result in less storage space needed by the consumer, thereby allowing the consumer to purchase larger quantities of typically bulky products.

In the above description of the present invention, the array of compressible articles have been described in terms of a one by x array, where x is the number of compressible articles in a row. The number of compressible articles in a row can vary, depending on factors such as the size and type of the articles, to obtain packages of particular or desired sizes. In addition, the array of articles can be wider than one row, for example, the array may have two, three or more rows.

In addition, the articles in the package of the present invention may or may not be folded. If the articles are folded, common folding techniques include the U-fold, a spiral fold, a W-fold. Other folding techniques include, a tri-fold, where the article is folded in thirds, a quad-fold, where the article is twice folded in half, or folded into quarters. The method of folding or the folding configuration is not critical to the present invention. Common folding means are describe in various patent publications, such as U.S. Patent Application Publication 2004/0168947 to McDonald, which is hereby incorporated by reference in its entirety.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A compressed package of articles comprising
a packaging component having a seal, and
a plurality of compressible articles in an array, the array of compressible articles having an uncompressed length, the compressible articles being compressed to a first compressed state having a first compressed length and disposed within the packaging component in the first compressed state, the packaging component retains the plurality of compressible articles in the first compressed state, and the packaging component and the plurality of compressible articles in the first compressed state form a package having a first package volume;
wherein the packaging component and the plurality of compressible articles in the first compressed state disposed in the packaging component are compressed together by vacuum compression to a second compressed state having a second package volume, the array of compressible articles in the second compressed state has a second compressed length, and the package is sealed with the seal such that the seal holds the package containing the plurality of compressible articles in the second compressed state, and the second compressed length of the array is less than the first compressed length of the array.

2. The compressed package of articles of claim 1, wherein the compressible articles in the first compressed state are compressed to the first compressed state by mechanical compression.

3. The compressed package of articles of claim 2, wherein when the seal is broken on the package containing the plurality of articles in the second compressed state, the package returns approximately to the first package volume and the array of compressible articles returns approximately to the first compressed length.

4. The compressed package of articles of claim 1, wherein the package component comprises an gas impermeable material.

5. The compressed package of articles of claim 4, wherein the gas impermeable material comprises a film.

6. The compressed package of articles of claim 1, wherein the first compressed length of the array is approximately between about 60% and about 95% of the uncompressed length of the array and the second compressed length is between about 50% and 90% of the first compressed length of the array.

7. The compressed package of articles of claim 6, wherein the first compressed length of the array is approximately between about 65% and about 80% of the uncompressed length of the array and the second compressed length of the array is between about 60% and about 80% of the first compressed length of the array and the second compressed length of the array is between about 40% and about 60% of the uncompressed length of the array.

8. The compressed package of articles of claim 1, wherein the articles comprise personal care articles.

9. The compressed package of articles of claim 8, wherein the personal care articles comprise diapers, training pants, adult incontinence pads, adult incontinence garments, sanitary napkins or pantiliners.

10. The compressed package of articles of claim 1, wherein the compressible articles in the first compressed state are compressed to the first compressed state by mechanical compression, wherein when the seal is removed from the package containing the plurality of articles in the second compressed state, the array will return to the first compressed length, the first compressed length of the array is approximately between about 65% and about 80% of the uncompressed length of the array and the second compressed length of the array is between about 60% and about 80% of the first compressed length of the array and the second compressed length of the array is between about 40% and about 60% of the uncompressed length of the array, the package component comprises an gas impermeable material, and the compressible articles comprise diapers, training pants, adult incontinence pads, adult incontinence garments, sanitary napkins or pantiliners.

11. The compressed package of articles of claim 2, further comprising an outer package, wherein the other package surrounds the compressed package of articles in the second compressed state.

12. The compressed package of articles of claim 1, further comprising an opening device which opens the seal to return the package of articles to the first compressed state.

13. The compressed package of articles of claim 10, further comprising an opening device which opens the seal to return the package of articles to the first compressed state.

14. A method of packaging a plurality of compressible articles, said method comprising
providing a packaging material;
providing an array of compressible articles, said array comprising a plurality of compressible articles and said array has an uncompressed length;
mechanically compressing the array of compressible articles to form a compressed array of compressible articles having a first compressed length;
forming a packaging component from the packaging material;
placing the compressed array of compressible articles having the first compressed length into the packaging component such that the array of compressible articles maintain the first compressed length;
compressing the compressed array of compressible articles in the packaging component by subjecting the compressed array of compressible articles in the packaging component to a pressure lower than atmospheric pressure, such that the array of compressible articles have a second compressed length; and
sealing the packaging component with a seal to form a sealed package of a compressed array of compressible articles.

15. The method of claim 14, wherein said forming the packaging component comprises vacuum forming the packaging component to a size which will retain the compressible articles in the first compressed state.

16. The method of claim 14, wherein the mechanical compression compresses the array of compressible articles to the first compressed length which is approximately between about 60% and about 95% of the uncompressed length of the array and vacuum compression compresses the array of compressible articles to a second compressed length which is approximately about 50% and about 90% of the first compressed length of the array.

17. The method of claim 16, wherein the mechanical compression compresses the array of compressible articles to the first compressed length which is approximately between about 65% and about 80% of the uncompressed length of the array and vacuum compression, compresses the array of compressible articles to a second compressed length which is approximately about 60% and about 80% of the first compressed length of the array.

18. The method of claim 17, wherein the mechanical compression and the vacuum compression combine to compress the array of compressible articles to the second compressed length of the array which is approximately between about 40% and about 60% of the uncompressed length of the array.

19. The method of claim 14, wherein the mechanical compression compresses the array of compressible article such that the array in the first compressed state exerts a force on the packing component of less than 100 psi.
